(19) United States
(12) Patent Application Publication
(10) Pub. No.: US 2026/0341497 A1
(43) Pub. Date: Oct. 29, 2026

(54) ARTIFICIAL INTELLIGENCE (AI) AGENT FOR OPERATING ROOM

(71) Applicant: ALCON INC., Fribourg (CH)

(72) Inventors: Aakash NAGARAJAN, Arlington, TX (US); Lu Yin, Fort Worth, TX (US); Nikhil Scaria, Fort Worth, TX (US); Ramesh Sarangapani, Coppell, TX (US); Sinchan Bhattacharya, Fort Worth, TX (US); Vignesh Suresh, Fort Worth, TX (US); Zhuoran Wu, Rancho Cucamonga, CA (US)

(21) Appl. No.: 19/645,082

(22) Filed: Apr. 10, 2026

Related U.S. Application Data
(60) Provisional application No. 63/794,865, filed on Apr. 25, 2025.

(51) Int. Cl. G16H 40/63; G06V 10/82

(52) U.S. Cl. CPC G16H 40/63; G06V 10/82; G06V 2201/03

(57) ABSTRACT

A method of managing medical procedures may include performing artificial-intelligence inference on image data captured during a medical procedure to obtain current contextual data about the medical procedure. The method may also include processing the current contextual data to determine an operational action in the context of the medical procedure and directing adjustments to operations of one or more medical devices based on the determined operational action.

### CROSS-REFERENCE TO RELATED APPLICATION

This application claims priority to U.S. Provisional Application No. 63/794,865, filed on Apr. 25, 2025, which is incorporated herein in its entirety.

### FIELD

The embodiments discussed herein relate to an artificial intelligence (AI) agent for an operation room.

### BACKGROUND

Some modern surgical procedures involve sophisticated medical equipment and complex workflows requiring coordination between surgical teams and various devices. For example, an operating room may include multiple interconnected systems including surgical microscopes, robotic instruments, monitoring equipment, and visualization displays. As a result, surgeons performing procedures may simultaneously be monitoring patient parameters, controlling surgical instruments, adjusting equipment settings, and/or documenting procedural steps.

The subject matter claimed herein is not limited to embodiments that solve any disadvantages or that operate only in environments such as those described above. Rather, this background is only provided to illustrate one example technology area where some embodiments described herein may be practiced.

### SUMMARY

According to an aspect of at least one embodiment, a method of managing medical procedures may include performing artificial-intelligence inference on image data captured during a medical procedure to obtain current contextual data about the medical procedure. The method may also include processing the current contextual data to determine an operational action in the context of the medical procedure and directing adjustments to operations of one or more medical devices based on the determined operational action.

The object and advantages of the embodiments will be realized and achieved at least by the elements, features, and combinations particularly pointed out in the claims. It is to be understood that both the foregoing general description and the following detailed description are explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1 illustrates a block diagram of an environment to manage medical procedures;

FIG. 2 illustrates a flowchart of an example method of managing medical procedures;

FIG. 3 is a block diagram of an example computing system for generative artificial intelligence (AI); and

FIG. 4 is a block diagram of an example computing device all in accordance with at least some embodiments of the present disclosure.

### DETAILED DESCRIPTION

Modern surgical procedures present significant challenges to surgical teams, requiring simultaneous management of complex medical devices, continuous monitoring of multiple parameters, precise instrument manipulation, and/or comprehensive documentation. As a result, surgeons and operating room personnel face substantial cognitive load as they coordinate these tasks while maintaining focus on surgical decisions and patient safety. The mental burden of tracking device settings, monitoring physiological parameters, retrieving instruments, and/or documenting procedural steps may lead to fatigue, reduced efficiency, and increased risk of complications. Additionally, valuable information displayed on device screens or embedded in surgical video feeds may be overlooked when surgeons concentrate on manual tasks, potentially resulting in delayed responses to concerning conditions.

The embodiments described herein address these challenges by providing an artificial intelligence (AI)-powered surgical management system that performs one or more actions as an intelligent surgical assistant. For example, the system may perform AI inference on image data captured during a medical procedure to obtain current contextual data about the medical procedure.

In some embodiments, the image data may be captured by a sensor or medical device and may include images of tissue and/or medical instruments involved in the procedure. In these and other embodiments, the current contextual data may include information regarding a phase of the medical procedure, tissue response of a patient undergoing the medical procedure, an anatomical structure of the patient, environmental conditions of the medical procedure, and/or conditions of medical instruments involved in the medical procedure.

The system may process the current contextual data to determine an operational action in the context of the medical procedure. The operational action may involve adjustments to the surgical procedure and/or to settings of medical devices being used. The system may then direct adjustments to operations of one or more medical devices based on the determined operational action.

By determining and directing adjustments to medical devices during a surgery, the system may reduce cognitive burden on surgeons. As a result, the system may enable surgeons to focus on decision-making and surgical execution rather than routine monitoring and device management tasks.

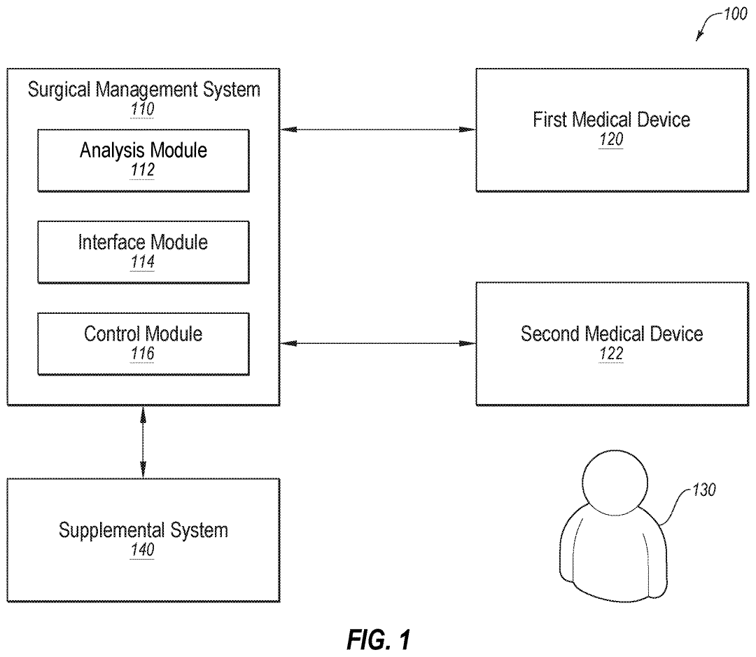

Referring now to FIG. 1, an environment 100 for managing medical procedures through artificial intelligence-powered surgical assistance may be illustrated, according to one or more embodiments. The environment 100 may include a surgical management system 110, a first medical device 120, a second medical device 122, a user 130, and a supplemental system 140. The surgical management system 110 may include an analysis module 112, an interface module 114, and a control module 116. In general, each component within the environment 100 may operate cooperatively to provide real-time surgical assistance during medical procedures.

In some embodiments, any operation performed by a module in this disclosure may be implemented in software, firmware, or a combination thereof. When implemented in software, the module may comprise one or more executable instructions stored on a non-transitory computer-readable medium and configured, when executed by one or more processors, to perform the operations described herein for that module. In this manner, the functionality attributed to the module may be carried out entirely through software execution, hardware execution, or any suitable combination of the two.

The environment 100 may implement network infrastructure for connecting all components. The network infrastructure may include wired Ethernet connections for high-bandwidth, low-latency communication. Alternately or additionally, the network infrastructure may include wireless access points for connecting mobile devices or wireless-enabled equipment. In some embodiments, the network infrastructure may include network switches with quality-of-service capabilities for prioritizing time-critical data streams.

The first medical device 120 may include a medical device that may be used to perform one or more aspects of a surgical procedure. In some embodiments, the first medical device 120 may be configured to interact with a patient undergoing the surgical procedure. For example, the first medical device 120 may be configured to gather data about the patient and/or include a device that interacts directly with the patient.

In some embodiments, the first medical device 120 may include one or more sensors and may be configured to generate sensor data that may be provided to the surgical management system 110. In some embodiments, the sensors may be imagers configured to provide images and/or a video feed of the surgical field. For example, the first medical device 120 may capture high-definition video at frame rates between thirty and sixty frames per second. As an example, the first medical device 120 may include a surgical microscope with integrated camera systems that may provide a video feed.

In some embodiments, the sensors of the first medical device 120 may be configured to obtain other data. For example, the sensor of the first medical device 120 may be configured to detect one or more of temperature, pressure, flow rate, position, orientation, force, torque, electrical impedance, optical properties, acoustic properties, chemical composition, biological markers, or environmental conditions relevant to the medical procedure being performed by the first medical device 120.

The first medical device 120 may transmit the sensor data to the surgical management system 110 through direct connections or network protocols.

The first medical device 120 may be further configured to perform functions for one or more surgical procedures. For example, the first medical device 120 may implement phacoemulsification functionality for cataract surgery procedures, generate ultrasonic vibrations for emulsifying lens material, control aspiration systems for removing emulsified tissue, regulate irrigation systems for maintaining intraocular pressure, and adjust ultrasonic power levels, among other surgical procedures. In some embodiments, the first medical device 120 may also be configured to perform functions for other surgical procedures beyond ophthalmic applications, including surgical procedures with respect to laparoscopic procedures, arthroscopic procedures, cardiovascular interventions, neurosurgical procedures, and other minimally invasive or open surgical techniques.

In some embodiments, the first medical device 120 may perform the functions based on control commands received from the surgical management system 110 and/or the user 130. For example, based on control commands, the first medical device 120 may begin or stop a function. In these and other embodiments, how the functions are performed by the first medical device 120 may be controlled by parameters that may be adjusted by the user 130 manually or may be adjusted via the surgical management system 110. For example, the surgical management system 110 may direct adjustments to parameters to the first medical device 120. In these and other embodiments, the adjusted parameters may be implemented automatically by the first medical device 120 in response to receiving direction from the surgical management system 110.

In some embodiments, examples of parameters that may be controlled may include cutting power levels for electrosurgical units, insufflation pressure for laparoscopic procedures, irrigation flow rates for arthroscopic procedures, radiofrequency energy delivery for cardiac ablation procedures, ultrasonic power for tissue dissection, vacuum levels for surgical suction devices, or temperature settings for thermal ablation systems, among other parameters.

In some embodiments, the first medical device 120 may provide video simultaneously to the surgical management system 110 and to display systems viewed by the user 130. In these and other embodiments, the display system may be part of the first medical device 120 or a separate unit. In these and other embodiments, the first medical device 120 may overlay data onto captured video frames. The overlaid data may include information regarding the surgical procedure being performed. In these and other embodiments, the data may result from sensor data captured by the first medical device 120 or another device. As an example, the overlay data may include intraocular pressure measurements, depth measurements indicating instrument penetration, flow rate indicators for irrigation and aspiration systems, power level displays for ultrasonic or laser energy sources, among other data.

The second medical device 122 may include a medical device that may be used to perform one or more aspects of a surgical procedure. In some embodiments, the second medical device 122 may be similar to the first medical device 120. Alternatively or additionally, the second medical device 122 may be different from the first medical device 120. For example, in some embodiments, the second medical device 122 may be configured to assist the user 130 without physical interaction with tissue of a patient undergoing the medical procedure. For example, the second medical device 122 may include robotic actuators for positioning surgical instruments or adjusting device parameters. In these and other embodiments, the second medical device 122 may include motorized arms with multiple degrees of freedom. Alternately or additionally, the second medical device 122 may retrieve instruments from storage locations and present them to the user 130 and/or may return used instruments to designated storage positions. Alternately or additionally, the second medical device 122 may adjust camera angles or positions and/or may modify lighting configurations.

In some embodiments, the second medical device 122 may perform functions based on control commands received from the surgical management system 110 and/or the user 130. For example, based on control commands, the second medical device 122 may begin or stop a function. In these and other embodiments, how the functions are performed by the second medical device 122 may be controlled by parameters that may be adjusted by the user 130 manually or may be adjusted via the surgical management system 110. For example, the surgical management system 110 may direct adjustments to parameters to the second medical device 122. In these and other embodiments, the adjusted parameters may be implemented automatically by second medical device 122 in response to receiving direction from the surgical management system 110.

In some embodiments, the second medical device 122 may implement sensors for detecting user interactions. The sensors may include force sensors detecting when the user 130 grasps instruments held by robotic arms. The sensors may include proximity sensors detecting when the user 130 approaches the device. The sensors may include position encoders tracking the current state of all motorized components. The second medical device 122 may report sensor data to the surgical management system 110 for logging and analysis. The second medical device 122 may adjust its behavior based on detected user interactions, such as relaxing grip force when the user 130 attempts to remove an instrument.

In some embodiments, the user 130 may be a surgeon or other medical professional performing the surgical procedure. The user 130 may interact with the first medical device 120, the second medical device 122, and the surgical management system 110. In these and other embodiments, the user 130 may receive interact from the surgical management system 110 through multiple modalities. The user 130 may hear verbal alerts and confirmations through audio output systems. The user 130 may view visual displays presenting parameter values, recommendations, or graphical overlays. The user 130 may feel haptic feedback through connected interface devices.

In some embodiments, the surgical management system 110 may serve as central hub for coordinating the real-time surgical assistance. In general, to achieve the real-time surgical assistance, the surgical management system 110 may process multiple data streams during a surgical procedure. For example, the surgical management system 110 may receive image data from one or more medical devices positioned within an operating room environment. The surgical management system 110 may execute artificial intelligence inference operations on the received image data to extract contextual information about ongoing surgical activities. After obtaining the contextual information, the surgical management system 110 may determine appropriate operational actions based on the extracted contextual information and may direct adjustments to connected medical devices based on the determined operational actions.

The surgical management system 110 may execute on computing hardware positioned within or adjacent to the operating room. The computing hardware may include one or more central processing units and/or one or more graphics processing units for accelerating artificial intelligence inference. In these and other embodiments, the computing hardware may include memory for storing instructions for execution, loading neural network models, and buffering video streams. Alternately or additionally, the computing hardware may include solid-state storage to store model parameters and configuration data. The computing hardware may also include network interface cards for communication with medical devices and external systems.

In some embodiments, the computing hardware may comprise edge computing devices configured for real-time inference. The edge computing devices may include specialized accelerators for neural network operations, provide hardware-level security features for protecting sensitive medical data, and may support remote management and software updates.

In some embodiments, the analysis module 112 within the surgical management system 110 may analyze the data captured during the medical procedures. For example, the surgical management system 110 may obtain data from the first medical device 120 and direct the data to the analysis module 112. In these and other embodiments, the analysis module may analyze the data to obtain current contextual data about the medical procedure.

In some embodiments, the current contextual data may broadly encompass information regarding one or more aspects of the medical procedure being performed. In some embodiments, the current contextual data may include information about a phase of the medical procedure, such as an identification of a current surgical step or stage within a predefined sequence of procedural steps. In some embodiments, the current contextual data may include information about tissue response of a patient undergoing the medical procedure, such as indicators of tissue stress, edema, hemorrhage, or other physiological reactions observable in the image data. In some embodiments, the current contextual data may include information about an anatomical structure of the patient, such as identification of specific organs, tissues, vessels, or other anatomical features visible in the surgical field. In some embodiments, the current contextual data may include information about environmental conditions of the medical procedure, such as lighting conditions, visibility quality, presence of fluids or obscurants, or positioning of surgical equipment.

In some embodiments, the current contextual data may include information about conditions of medical instruments involved in the medical procedure, such as instrument type, position, orientation, depth of insertion, proximity to critical structures, or operational status. In some embodiments, the current contextual data may include information about device parameters or settings, such as pressure levels, flow rates, energy delivery settings, or other operational parameters of medical devices being used during the procedure. In some embodiments, the current contextual data may include information about temporal aspects of the procedure, such as elapsed time, duration of current phase, or timing relative to critical events.

In some embodiments, the current contextual data may include combinations of multiple types of information, providing a comprehensive understanding of the procedural state at a given moment.

In some embodiments, the analysis module 112 may employ artificial intelligence inference to obtain the current contextual data from the received data. For example, in some embodiments, the data may be image or video data. In these and other embodiments, the analysis module 112 may utilize machine learning models trained on medical imaging datasets to identify anatomical structures, surgical instruments, and procedural phases within the image data. For example, the analysis module 112 may apply convolutional neural networks to detect and classify objects visible in surgical video frames, such as surgical tools, tissue types, or anatomical landmarks. The analysis module 112 may employ natural language processing algorithms to extract numerical values and textual information from overlay data embedded within the image data, such as pressure readings, flow rates, or device status indicators displayed on surgical equipment screens.

In some embodiments, the analysis module 112 may utilize temporal analysis techniques to track changes in tissue appearance over time, enabling detection of tissue responses such as edema formation, hemorrhage development, or wound healing progression. Alternately or additionally, the analysis module 112 may apply computer vision algorithms to measure dimensions of anatomical structures, such as incision lengths, capsulotomy diameters, or implant positions, providing quantitative assessments of surgical precision. Alternately or additionally, the analysis module 112 may employ pattern recognition techniques to identify surgical phases by analyzing sequences of actions, instrument usage patterns, and anatomical changes observed across multiple frames of the image data. Alternately or additionally, the analysis module 112 may utilize depth estimation algorithms to determine three-dimensional spatial relationships between surgical instruments and anatomical structures, enabling assessment of instrument proximity to critical tissues. Alternately or additionally, the analysis module 112 may apply anomaly detection algorithms to identify deviations from expected surgical progressions, such as unexpected bleeding, instrument malpositions, or tissue damage, generating alerts for potentially concerning conditions.

In some embodiments, the data captured during the medical procedures may be data other than image data. For example, the data may include sensor readings from medical instruments, such as temperature measurements from thermal probes, force measurements from surgical forceps, electrical impedance measurements from tissue contact sensors, or flow rate measurements from irrigation systems. In these and other embodiments, the analysis module 112 may apply machine learning models trained on sensor data to identify patterns indicative of specific tissue conditions or procedural states. For example, the analysis module 112 may utilize time-series analysis algorithms to detect trends in temperature data that correlate with tissue coagulation or thermal damage, enabling early warning of potential complications. Alternately or additionally, the analysis module 112 may employ anomaly detection algorithms on force sensor data to identify unusual resistance patterns that may indicate unexpected tissue density, adhesions, or instrument malfunction. Alternately or additionally, the analysis module 112 may apply signal processing techniques to electrical impedance measurements to characterize tissue types, such as distinguishing between healthy tissue, scar tissue, or tumor tissue based on impedance signatures. Alternately or additionally, the analysis module 112 may utilize regression models to predict irrigation flow rates based on current tissue hydration levels measured by optical sensors or acoustic sensors.

In some embodiments, the surgical management system 110 may select and apply algorithms based on multiple factors including the type of surgical procedure being performed, the current phase or stage within that procedure, and data previously collected during the procedure or from preoperative assessments. In these and other embodiments, the surgical management system 110 may maintain a library of algorithms for different surgical contexts. For example, during a phacoemulsification procedure, the surgical management system 110 may initially apply algorithms trained to detect corneal incision characteristics and capsulotomy parameters. As the procedure progresses to the phacoemulsification phase, the surgical management system 110 may transition to algorithms specialized in detecting lens density, monitoring ultrasound tip position relative to the posterior capsule, and assessing aspiration flow patterns.

In some embodiments, the selection process may be informed by the current surgical phase identified by the analysis module 112 through video analysis. The surgical management system 110 may also consider patient-specific data collected preoperatively, such as optical coherence tomography scans indicating lens density or anterior chamber depth measurements, to select algorithms with appropriate sensitivity thresholds.

In some embodiments, during the procedure, the surgical management system 110 may continuously evaluate the performance of active algorithms and may switch to alternative algorithms if conditions change unexpectedly. For instance, in response to the video analyzing component detecting unusual tissue responses or complications, the surgical management system 110 may activate specialized algorithms trained to monitor for specific complication indicators.

In some embodiments, the algorithm selection may also be influenced by surgeon preferences stored in user profiles, where certain surgeons may prefer more conservative alert thresholds while others may prefer more aggressive parameter optimization. The surgical management system 110 may log all algorithm selections and transitions, creating a record of which analytical approaches were applied at each stage of the procedure. This algorithmic adaptability may enable the surgical management system 110 to provide contextually appropriate assistance throughout the entire surgical workflow.

In some embodiments, the analysis module 112 may provide the current contextual data to the control module 116.

In some embodiments, the interface module 114 may manage bidirectional communication between the user 130 and the surgical management system 110. The interface module 114 may manage the bidirectional communication through one or more interaction modalities, including voice-based communication, visual presentation, and tactile feedback. Each modality may enable the user 130 to interact with the surgical management system 110 in different contexts and for different purposes.

For example, in some embodiments, the interface module 114 may process voice commands spoken by the user 130 during surgical procedures. For example, the interface module 114 may receive an audio signal corresponding to a spoken command such as “display patient vitals” or “zoom in on the surgical field,” convert the audio signal into a text transcription, and interpret the intent of the transcribed voice command to trigger an appropriate system response. The interface module 114 may also generate verbal responses to user queries. For example, when the user 130 asks “what was the last recorded blood pressure,” the interface module 114 may retrieve the relevant data and synthesize speech from a text response such as “the last recorded blood pressure was 120 over 80 millimeters of mercury.”

In some embodiments, the interface module 114 may present visual information to the user 130 through display systems. For example, the interface module 114 may overlay graphical elements onto surgical video feeds, such as anatomical labels, instrument tracking indicators, or real-time measurement annotations, to augment the visual information available to the user 130 during a procedure.

In some embodiments, the interface module 114 may provide haptic feedback through connected interface devices. For example, the interface module 114 may cause a handheld controller or surgical instrument to vibrate or apply resistance when the user 130 approaches a predefined boundary or safety zone within the surgical field.

In some embodiments, the interface module 114 may generate confirmation requests when the surgical management system 110 identifies conditions requiring user 130 approval before taking action. The confirmation requests may describe a detected condition, explain a recommended action and an expected outcome associated with the recommended action, present one or more options for surgeon selection, and specify whether a situation requires an immediate response. The interface module 114 may await confirmation from the user 130 before proceeding with a recommended action. The interface module 114 may accept an affirmative response, a negative response, or a modified instruction from the user 130.

The control module 116 may be configured to obtain the current contextual data from the analysis module 112 and determine an operation action in the context of the medical procedure. In some embodiments, the operational action may be determined by evaluating the current contextual data against predefined criteria, thresholds, or decision rules stored within the control module 116. In these and other embodiments, the rules may be defined based on clinical guidelines, standard operating procedures, and/or surgeon preferences.

As an example, the control module 116 may compare measured intraocular pressure values extracted from the current contextual data to predetermined safe pressure ranges for the specific surgical phase identified by the analysis module 112. In some embodiments, the control module 116 may utilize lookup tables that map combinations of surgical phase, tissue conditions, and device parameters to recommended operational actions. For example, a lookup table entry may specify that during phacoemulsification phase with detected high lens density and current ultrasound power below a threshold value, the recommended operational action may be to increase ultrasound power by a specified increment. As another example, a rule may specify that when aspiration tip proximity to posterior capsule falls below a minimum safe distance threshold, the operational action may be to reduce vacuum level to prevent capsular rupture.

In some embodiments, the control module 116 may utilize machine learning models trained on historical surgical data to predict optimal operational actions for given combinations of current contextual data. For example, a trained model may predict that for a patient with specific preoperative characteristics undergoing a particular surgical phase with observed tissue responses, a particular adjustment to irrigation flow rate may optimize surgical outcomes.

In some embodiments, the operational action determined by the control module 116 may include adjustments to device parameters such as ultrasound power levels, aspiration rates, vacuum pressures, irrigation flow rates, or other controllable settings of the first medical device 120 or second medical device 122. In some embodiments, the operational action may include instructions to robotic systems to adjust positions, orientations, or engagement states of surgical instruments or equipment.

In some embodiments, the operational action may include generation of alerts or recommendations to be presented to the user 130 via the interface module 114. In some embodiments, the operational action may include commands to external systems such as documentation systems or imaging systems to capture specific data or update records.

In some embodiments, the operational actions determined by the control module 116 may result in adjustments to how the first medical device 120 and/or the second medical device 122 operate during the medical procedure. In these and other embodiments, the control module 116 may direct the adjustments to the first medical device 120 and/or the second medical device 122. For example, the control module 116 may send a command to the first medical device 120 to adjust a flow rate of an aspirator of the first medical device 120.

In some embodiments, the control module 116 may direct adjustments to operations automatically. Alternately or additionally, the control module 116 may direct adjustments to operations only after confirmation from the user 130.

Alternately or additionally, the control module 116 may direct adjustments to operations based on the determined operational actions through various execution strategies depending on system configuration, procedural context, and safety considerations. For example, in some embodiments, the control module 116 may automatically implement certain operational actions without confirmation from the user 130 and may wait for confirmation from the user 130 before implementing other operational actions.

In some embodiments, the determination of which operational actions require confirmation may be based on predefined criteria stored within the control module 116. For example, the control module 116 may automatically implement minor parameter adjustments that fall within predetermined safe ranges, such as small incremental changes to irrigation flow rates or modest adjustments to vacuum levels that remain well below safe thresholds. Alternately or additionally, the control module 116 may require confirmation from the user 130 for operational actions that involve larger parameter changes, such as increases in ultrasound power levels above a threshold or adjustments to aspiration rates that approach safety limits. Alternately or additionally, the control module 116 may require confirmation for actions that involve changes to multiple device parameters simultaneously and/or actions that affect surgical phases where precision and surgeon oversight are particularly important.

In some embodiments, the interface module 114 may present confirmation requests to the user 130 when the control module 116 determines that confirmation is required, and the control module 116 may await an affirmative response from the user 130 before directing adjustments to a target medical device.

In some embodiments, the surgical management system 110 may adapt behavior based on the current surgical phase. In these and other embodiments, the analysis module 112 may identify surgical phases from video analysis of the surgical field. For example, during cataract surgery, the analysis module 112 may identify phases such as corneal incision, capsulotomy, phacoemulsification, irrigation/aspiration, and intraocular lens implantation based on visual characteristics of the surgical field, instrument types detected in the video data, and temporal sequences of observed actions. The control module 116 may adjust monitoring priorities based on the identified phase. For example, during the capsulotomy phase, the control module 116 may prioritize monitoring of capsule integrity and capsulotomy diameter measurements, while during the phacoemulsification phase, the control module 116 may prioritize monitoring of ultrasound tip proximity to the posterior capsule and lens fragment density.

In some embodiments, the control module 116 may prepare instruments or adjust device settings in anticipation of upcoming phases. For example, after detecting completion of the phacoemulsification phase, the control module 116 may direct the second medical device 122 to position irrigation/aspiration instruments for retrieval by the user 130 or may adjust irrigation flow rates in preparation for the irrigation/aspiration phase.

In some embodiments, the control module 116 may modify alert sensitivity based on phase-specific risk factors. For example, during phases involving proximity to critical structures such as the posterior capsule, the control module 116 may increase alert sensitivity for instrument depth measurements and vacuum pressure levels. The interface module 114 may provide phase-appropriate recommendations to the user 130, such as suggesting parameter adjustments tailored to the current phase or displaying phase-specific guidance overlays on the surgical microscope display.

In some embodiments, the surgical management system 110 may learn from historical surgical data to improve performance over time. In these and other embodiments, the surgical management system 110 may track which recommendations are accepted or rejected by the user 130 during surgical procedures. For example, when the control module 116 generates a recommendation to adjust a device parameter and presents the recommendation to the user 130 via the interface module 114, the surgical management system 110 may record whether the user 130 accepts the recommendation, rejects the recommendation, or modifies the recommendation before implementation. The control module 116 may adjust recommendation thresholds based on surgeon preferences derived from the tracked acceptance and rejection patterns.

In some embodiments, the surgical management system 110 may identify patterns in successful surgical outcomes by analyzing correlations between device parameter settings, surgical phase durations, tissue response indicators, and postoperative outcome data. The control module 116 may refine lookup tables based on accumulated experience, such as updating optimal parameter ranges for specific combinations of patient characteristics, surgical phases, and tissue conditions when historical data indicates that particular parameter settings correlate with improved outcomes. The surgical management system 110 may aggregate learning across multiple procedures and multiple surgeons to continuously improve the accuracy of recommendations.

In some embodiments, the supplemental system 140 may include external software applications or services that may interact with the surgical management system 110. For example, the supplemental system 140 may include note-taking applications for surgical documentation.

As an example of operation of the environment 100, the surgical management system 110 may implement a workflow for laser-assisted cataract surgery procedures. The workflow may provide preoperative data integration, automated laser parameter recommendation, dynamic adjustment, and visualization enhancement. For the preoperative data integration, the analysis module 112 may analyze optical coherence tomography (OCT) scan data of a patient to assess cataract density and pupil size. The analysis module 112 may extract quantitative measurements from the OCT scan data, such as lens opacity values, anterior chamber depth measurements, and pupil diameter measurements. Based on the preoperative data and real-time video analysis of the cataract, the control module 116 may proactively generate laser parameter settings. The interface module 114 may present the recommended laser parameter settings to the user 130 via a heads-up display, such as displaying “Recommended energy: 15%, Spot Size: 0.8 mm.” For dynamic adjustment during the procedure, the analysis module 112 may continuously analyze tissue response as the user 130 progresses through the surgical phases. The control module 116 may automatically adjust laser parameters in real-time to increase fragmentation and reduce energy delivery. For example, the control module 116 may reduce laser energy levels when the analysis module 112 detects tissue stress indicators in the video data or may increase spot size when lens density measurements indicate harder cataract material. For visualization enhancement, the interface module 114 may overlay a virtual guide on the surgical microscope display or direct the first medical device 120 to present the overlay. The virtual guide may show an optimal capsulotomy diameter and centering based on patient anatomy extracted from the preoperative OCT scan data and real-time video analysis. The virtual guide may include concentric circles indicating target capsulotomy boundaries, alignment markers for centering relative to the lens axis, and depth indicators for maintaining instrument positioning.

As another example of operation, the surgical management system 110 may implement a workflow for fragment removal and irrigation/aspiration phases of cataract surgery procedures. During fragment removal and irrigation/aspiration, the user 130 may manually adjust irrigation/aspiration parameters to remove cortical fragments, often relying on tactile feedback and visual assessment. The surgical management system 110 may provide fragment detection and automated irrigation/aspiration control.

For fragment detection, the analysis module 112 may utilize image processing algorithms to detect and track cortical fragments in real-time from video data captured by the first medical device 120. The analysis module 112 may identify fragment size, fragment location, and tissue consistency characteristics from the video data. Based on the fragment size, fragment location, and tissue consistency, the control module 116 may automatically adjust irrigation/aspiration parameters for removal and lower risk of complications. For example, the control module 116 may increase vacuum pressure for denser fragment removal when the analysis module 112 detects high-density cortical material or may decrease flow rate when fragments are detected near sensitive structures. For risk mitigation, the interface module 114 may provide alerts when vacuum pressure exceeds predetermined threshold values or when the aspiration tip approaches critical structures such as the posterior capsule or zonular fibers. The alerts may include visual indicators on the heads-up display, auditory signals, or haptic feedback to the user 130. To help with efficiency, the control module 116 may increase fragment removal by prioritizing areas with higher fragment density. The analysis module 112 may generate a fragment density map from the video data, and the interface module 114 may overlay the fragment density map on a display to guide the user 130 toward regions requiring attention. The control module 116 may adjust aspiration parameters based on the fragment density map to increase removal in high-density regions while maintaining safety margins in low-density regions.

Modifications, additions, or omissions may be made to the environment 100 without departing from the scope of the present disclosure. For example, the environment 100 may operate within an operating room containing additional equipment not explicitly illustrated in FIG. 1. For example, the environment 100 may include additional medical devices. Alternately or additionally, the operating room may include patient monitoring systems tracking vital signs, lighting systems for illuminating the surgical field, ventilation systems maintaining appropriate environmental conditions. The surgical management system 110 may integrate with some or all this additional equipment.

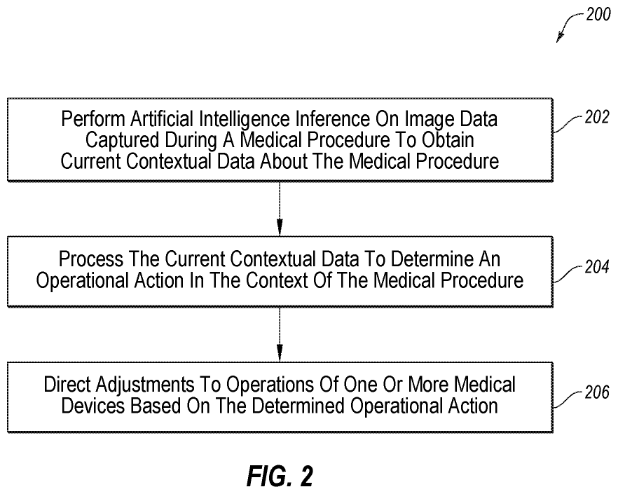

Referring now to FIG. 2, a method 200 of managing medical procedures may be illustrated, according to one or more embodiments. The method 200 may be performed by any suitable system, apparatus, or device. For example, the method 200 may be implemented by the surgical management system 110 described throughout the present disclosure. Although illustrated with discrete blocks, the steps and operations associated with one or more blocks of the method 200 may be divided into additional blocks, combined into fewer blocks, or eliminated, depending on the particular implementation.

At block 202, artificial intelligence inference may be performed on image data captured during a medical procedure to obtain current contextual data about the medical procedure. The image data may be captured by one or more sensors or medical devices positioned to visualize a surgical field during the medical procedure. In some embodiments, the artificial intelligence inference may be performed by an analysis module of a surgical management system.

In some embodiments, the current contextual data obtained from the artificial intelligence inference may include information regarding a phase of the medical procedure. In some embodiments, the current contextual data may include information about tissue response of a patient undergoing the medical procedure. Alternately or additionally, the current contextual data may include information about an anatomical structure of the patient. Alternatively or additionally, the current contextual data may include information about environmental conditions of the medical procedure. Alternately or additionally, the current contextual data may include information about conditions of medical instruments involved in the medical procedure and/or information about device parameters or settings.

In some embodiments, the artificial intelligence inference may be performed continuously throughout the medical procedure. For example, the artificial intelligence inference may be performed on each frame of the image data as the frame is received, at regular intervals, and/or in response to detection of a change in the surgical field. The change in the surgical field may be detected by comparing consecutive frames of the image data.

At block 204, the current contextual data may be processed to determine an operational action in the context of the medical procedure. The processing may be performed by a control module of the surgical management system. The control module may evaluate the current contextual data against predefined criteria.

In some embodiments, the operational action determined by the control module may include adjustments to device parameters, instructions to robotic systems to adjust positions of surgical instruments, and generation of alerts to be presented to a user via an interface module, among others.

At block 206, adjustments to operations of one or more medical devices may be directed based on the determined operational action. The directing of adjustments may be performed by the control module. The control module may generate device-specific command messages and may transmit the device-specific command messages to the one or more medical devices.

In some embodiments, the one or more medical devices that receive the adjustments to operations may include the medical devices that provided the image data during the medical procedure. Alternately or additionally, the one or more medical devices that receive the adjustments to operations may include medical devices different from the devices that provided the image data.

In some embodiments, the directing of adjustments may occur automatically without requiring explicit confirmation from a user. Alternately or additionally, the directing of adjustments may require confirmation from a user before implementation. In these and other embodiments, the control module may generate a confirmation request. The confirmation request may describe the detected condition that prompted the operational action and may describe the recommended adjustment. In these and other embodiments, an interface module may present the confirmation request to the user. The presentation may be verbal through synthesized speech, visual through text displayed, or otherwise using various means. The interface module may receive input from the user. After receiving affirmative confirmation, the control module may proceed with directing the adjustment to the medical device. In response to receiving denial, the control module may refrain from directing the adjustment and may log the denial for future reference. In response to receiving a modification, the control module may adjust the command parameters according to the modification and then direct the modified adjustment to the medical device. In some embodiments, the requirement for confirmation may depend on characteristics of the adjustment.

In some embodiments, the directing of adjustments may be iterative. For example, after directing an initial adjustment, the control module may continue to monitor the current contextual data obtained from ongoing artificial intelligence inference on subsequent image data. The control module may determine whether the initial adjustment achieved the desired effect. If the desired effect was not achieved, the control module may determine a subsequent operational action. The subsequent operational action may include a further adjustment to the same parameter or an adjustment to a different parameter. The control module may direct the subsequent adjustment to the medical device. The iterative process may continue until the desired surgical condition is achieved or until the user intervenes to halt the automated adjustments.

One skilled in the art will appreciate that, for this and other processes, operations, and methods disclosed herein, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments.

For example, in some embodiments, the method 200 may further include continually executing the blocks 202 and 204 during two or more phases of the medical procedure to provide ongoing adjustments to operations of the one or more medical devices during the two or more phases. The continual execution may involve repeating block 202 for each new frame or set of frames of image data received during the medical procedure. The continual execution may involve repeating block 204 each time new current contextual data is obtained. The continual execution may involve repeating block 206 each time a new operational action is determined. The ongoing adjustments may adapt to changing conditions throughout the medical procedure.

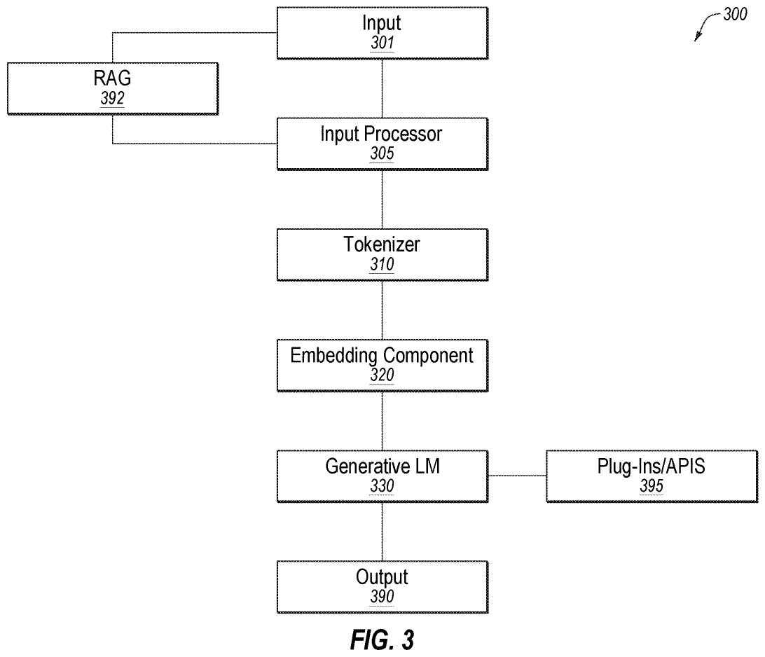

FIG. 3 is a block diagram of an example generative language model system 300 suitable for use in implementing at least some embodiments of the present disclosure. In the example illustrated in FIG. 3, the generative language model system 300 includes a retrieval augmented generation (RAG) component 392, an input processor 305, a tokenizer 310, an embedding component 320, plug-ins/APIs 395, and a generative language model (LM) 330 (which may include an LLM (large language model), a VLM (vision language model), a MMLM (multi-modal language model), a VLA (vision language action) model, etc.). The generative language model system 300 may be an example of a system that performs the AI models in the modules described with respect to FIG. 1.

At a high level, the input processor 305 may receive an input 301 comprising text and/or other types of input data (e.g., audio data, video data, frame data, sensor data), depending on the architecture of the generative LM 330 (e.g., LLM/LRM/VLM/MMLM/etc.). Taking raw input text as an example, the input processor 305 may prepare raw input text in various ways. For example, the input processor 305 may perform various types of text filtering to remove noise (e.g., special characters, punctuation, HTML (Hypertext Markup Language) tags, stopwords, portions of a frame(s), portions of audio, etc.) from relevant textual content.

In some embodiments, a RAG component 392 (which may include one or more RAG models, and/or may be performed using the generative LM 330 itself) may be used to retrieve additional information to be used as part of the input 301 or prompt. RAG may be used to enhance the input to the LLM/LRM/VLM/MMLM/etc. with external knowledge, so that answers to specific questions or queries or requests are more relevant—such as in a case where specific knowledge is required. The RAG component 392 may fetch this additional information (e.g., grounding information, such as grounding text/frame/video/audio/USD/CAD/etc.) from one or more external sources, which can then be fed to the LLM/LRM/VLM/MMLM/etc. along with the prompt to improve accuracy of the responses or outputs of the model.

For example, in some embodiments, the input 301 may be generated using the query or input to the model (e.g., a question, a request, etc.) in addition to data retrieved using the RAG component 392. In some embodiments, the input processor 305 may analyze the input 301 and communicate with the RAG component 392 (or the RAG component 392 may be part of the input processor 305, in embodiments) in order to identify relevant text and/or other data to provide to the generative LM 330. The RAG component 392 may use various RAG techniques such as, naïve RAG, modular RAG techniques, and/or Graph RAG.

The tokenizer 310 may segment the (e.g., processed) text data into smaller units (tokens) for subsequent analysis and processing. The tokens may represent individual words, subwords, characters, portions of audio/video/frame/etc., depending on the implementation. For example, the tokenizer 310 may convert the (e.g., processed) text into a structured format according to tokenization schema being implemented in the particular embodiment.

The embedding component 320 may use any known embedding technique to transform discrete tokens into (e.g., dense, continuous vector) representations of semantic meaning. For example, the embedding component 320 may use pre-trained word embeddings (e.g., Word2Vec, GloVe, or FastText), one-hot encoding, Term Frequency-Inverse Document Frequency (TF-IDF) encoding, one or more embedding layers of a neural network, and/or otherwise. In some implementations in which the input 301 includes video data, the input processor 301 may extract frames or apply resizing to extracted frames, and the embedding component 320 may extract features such as optical flow embeddings or video embeddings and/or may encode temporal information or sequences of frames.

The generative LM 330 and/or other components of the generative LM system 300 may use different types of neural network architectures depending on the implementation. For example, transformer-based architectures such as those used in models like GPT (Generative Pre-trained Transformer)may be implemented and may include self-attention mechanisms. Some other non-limiting example architectures include transformers (e.g., encoder-decoder, decoder only, multi-modal), RNNs (Recurrent Neural Network), LSTMs (Long Short-Term Memory), fusion models, diffusion models, cross-modal embedding models that learn joint embedding spaces, graph neural networks (GNNs), hybrid architectures combining different types of architectures adversarial networks like generative adversarial networks or GANs or adversarial autoencoders (AAEs) for joint distribution learning, linear-time sequence modeling with selective state space modeling (SSM) architectures (e.g., Mamba LLM architectures), and/or others. As such, depending on the implementation and architecture, the embedding component 320 may apply an encoded representation of the input 301 to the generative LM 330, and the generative LM 330 may process the encoded representation of the input 301 to generate an output 390, which may include responsive text and/or other types of data.

As described herein, in some embodiments, the generative LM 330 may be configured to access or use—or capable of accessing or using—plug-ins/APIs 395 (which may include one or more plug-ins, application programming interfaces (APIs), databases, data stores, repositories, etc.). These and other architectures for LLMs/VLMs/MMLMs/VLAs/etc. described herein are meant simply as examples, and other suitable architectures may be implemented within the scope of the present disclosure.

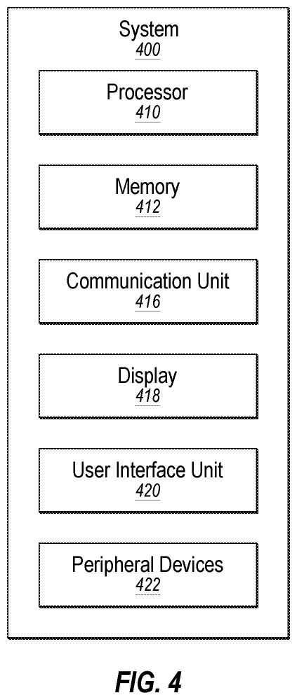

FIG. 4 illustrates an example system 400 that may be used during transcription of communications. The system 400 may be arranged in accordance with at least one embodiment described in the present disclosure. The system 400 may include a processor 410, memory 412, a communication unit 416, a display 418, a user interface unit 420, and a peripheral device 422, which all may be communicatively coupled. In some embodiments, the system 400 may be part of any of the systems or devices described in this disclosure. For example, the system 400 may be part of the surgical management system 110 of FIG. 1 and/or part of the first medical device 120 and/or the second medical device 122.

Generally, the processor 410 may include any suitable special-purpose or general-purpose computer, computing entity, or processing device including various computer hardware or software modules and may be configured to execute instructions stored on any applicable computer-readable storage media. For example, the processor 410 may include a microprocessor, a microcontroller, a parallel processor such as a graphics processing unit (GPU) or tensor processing unit (TPU), a digital signal processor (DSP), an application-specific integrated circuit (ASIC), a Field-Programmable Gate Array (FPGA), quantum computer, or any other digital or analog circuitry configured to interpret and/or to execute program instructions and/or to process data.

Although illustrated as a single processor in FIG. 4, it is understood that the processor 410 may include any number of processors distributed across any number of networks or physical locations that are configured to perform individually or collectively any number of operations described herein. In some embodiments, the processor 410 may interpret and/or execute program instructions and/or process data stored in the memory 412. In some embodiments, the processor 410 may execute the program instructions stored in the memory 412.

For example, in some embodiments, the processor 410 may execute program instructions stored in the memory 412 that are related to selecting a transcription generation technique for a communication session such that the system 400 may perform or direct the performance of the operations associated therewith as directed by the instructions. In these and other embodiments, the instructions may be used to perform one or more operations of the methods described in this disclosure.

The memory 412 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 410.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store particular program code in the form of computer-executable instructions or data structures and which may be accessed by a general-purpose or special-purpose computer. Combinations of the above may also be included within the scope of computer-readable storage media.

Computer-executable instructions may include, for example, instructions and data configured to cause the processor 410 to perform a certain operation or group of operations as described in this disclosure. In these and other embodiments, the term “non-transitory” as explained in the present disclosure should be construed to exclude only those types of transitory media that were found to fall outside the scope of patentable subject matter in the Federal Circuit decision of In re Nuijten, 500 F.4d 1446 (Fed. Cir. 2007). Combinations of the above may also be included within the scope of computer-readable media.

The communication unit 416 may include any component, device, system, or combination thereof that is configured to transmit or receive information over a network. In some embodiments, the communication unit 416 may communicate with other devices at other locations, the same location, or even other components within the same system. For example, the communication unit 416 may include a modem, a network card (wireless or wired), an infrared communication device, a wireless communication device (such as an antenna), and/or chipset (such as a Bluetooth device, an 802.6 device (e.g., Metropolitan Area Network (MAN)), a WiFi device, a WiMax device, cellular communication facilities, etc.), and/or the like. The communication unit 416 may permit data to be exchanged with a network and/or any other devices or systems described in the present disclosure.

The display 418 may be configured as one or more displays, like an LCD (liquid crystal display), LED (light emitting diode), OLED (organic light emitting diode), Braille terminal, or other type of display. The display 418 may be configured to present video, text captions, user interfaces, and other data as directed by the processor 410. The user interface unit 420 may include any device to allow a user to interface with the system 400. For example, the user interface unit 420 may include a mouse, a track pad, a keyboard, buttons, camera, and/or a touchscreen, among other devices. The user interface unit 420 may receive input from a user and provide the input to the processor 410. In some embodiments, the user interface unit 420 and the display 418 may be combined.

The peripheral devices 422 may include one or more devices. For example, the peripheral devices may include a microphone, a framer, and/or a speaker, among other peripheral devices. Modifications, additions, or omissions may be made to the system 400 without departing from the scope of the present disclosure. For example, in some embodiments, the system 400 may include any number of other components that may not be explicitly illustrated or described. Further, depending on certain implementations, the system 400 may not include one or more of the components illustrated and described.

As indicated above, the embodiments described herein may include the use of a special purpose or general-purpose computer (e.g., the processor 410 of FIG. 4) including various computer hardware or software modules, as discussed in greater detail below. Further, as indicated above, embodiments described herein may be implemented using computer-readable media (e.g., the memory 412 of FIG. 4) for carrying or having computer-executable instructions or data structures stored thereon.

Generally speaking, network environments suitable for use in implementing embodiments of the disclosure may include one or more client devices, servers, network attached storage (NAS), other backend devices, and/or other device types. The client devices, servers, and/or other device types (e.g., each device) may be implemented on one or more instances of the computing systems(s) 400 of FIG. 4—e.g., each device may include similar components, features, and/or functionality of the computing systems(s) 400. In addition, where backend devices (e.g., servers, NAS, etc.) are implemented, the backend devices may be included as part of a data center (such as, but not limited to, those described herein).

Components of a network environment may communicate with each other via a network(s), which may be wired, wireless, or both. The network may include multiple networks, or a network of networks. Compatible network environments may include one or more peer-to-peer network environments—in which case a server may not be included in a network environment—and one or more client-server network environments—in which case one or more servers may be included in a network environment. In peer-to-peer network environments, functionality described herein with respect to a server(s) may be implemented on any number of client devices.

In at least one embodiment, a network environment may include one or more cloud-based network environments, a distributed computing environment, a combination thereof, etc. A cloud-based network environment may be private (e.g., limited to a single organization), may be public (e.g., available to many organizations), and/or a combination thereof (e.g., a hybrid cloud environment).

The various features illustrated in the drawings may be, but are not necessarily, drawn to scale. The illustrations presented in the present disclosure are not meant to be actual views of any particular apparatus (e.g., device, system, etc.) or method but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” among others).

Relative terms used in the present disclosure and especially in the appended claims (e.g., bodies of the appended claims) are generally intended as falling within manufacturing tolerances and/or within scope reasonably understood by a person of skill in the art. For example, if two components are identified as being the “same” size, there may be variations consistent with manufacturing variances. Terms describing “approximately,” “similar,” “substantially,” or other terms designating similarity may convey within ten percent of the comparative value. For example, two components that are approximately the same size would be understood to be of a size within ten percent of each other.

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.” However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absent a showing that the terms “first,” “second,” “third,” etc., connote a specific order, these terms should not be understood to connote a specific order. Furthermore, absent a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited in the present disclosure are intended for pedagogical objects to aid the reader in understanding the invention and the concepts contributed by the inventor to furthering the art and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. A method of managing medical procedures, the method comprising:
performing artificial-intelligence inference on image data captured during a medical procedure to obtain current contextual data about the medical procedure;
processing the current contextual data to determine an operational action in the context of the medical procedure; and
directing adjustments to operations of one or more medical devices based on the determined operational action.

2. The method of claim 1, wherein the one or more medical devices that obtain the adjustments to operations are configured to provide the image data during the medical procedure.

3. The method of claim 1, further comprising:
presenting the operational action to medical personnel conducting the medical procedure; and
in response to presenting the operational action, obtaining input from the medical personnel, wherein the directing adjustments to operations of one or more medical devices occurs in response to the input from the medical personnel.

4. The method of claim 3, wherein the operational action is presented verbally, integrated with the image data, or presented separately on a display.

5. The method of claim 1, wherein the adjustments to operations of one or more medical devices are automatically implemented.

6. The method of claim 1, wherein the current contextual data includes one or more of: a phase of the medical procedure, tissue response of a patient undergoing the medical procedure, an anatomical structure of the patient, environmental conditions of the medical procedure, and conditions of medical instruments involved in the medical procedure.

7. The method of claim 1, wherein the adjustments to operations of one or more medical devices include one or more of: adjusting operational settings used during the medical procedure, overlaying information on image data presented by the one or more medical devices, and performing different operational functions.

8. The method of claim 1, further comprising continually executing the steps of performing and processing during two or more phases of the medical procedure to provide ongoing adjustments to operations of the one or more medical devices during the two or more phases.

9. A system comprising:
one or more computer-readable media configured to store instructions;
one or more processors coupled to the computer-readable media and configured to execute the instructions to cause or direct the system to perform operations, the operations comprising:
performing artificial-intelligence inference on image data captured during a medical procedure to obtain current contextual data about the medical procedure;
processing the current contextual data to determine an operational action in the context of the medical procedure; and
directing adjustments to operations of one or more medical devices based on the determined operational action.

10. The system of claim 9, wherein the one or more medical devices that obtain the adjustments to operations are configured to provide the image data during the medical procedure.

11. The system of claim 9, wherein the operations further comprise:
presenting the operational action to medical personnel conducting the medical procedure; and
in response to presenting the operational action, obtaining input from the medical personnel, wherein the directing adjustments to operations of one or more medical devices occurs in response to the input from the medical personnel.

12. The system of claim 9, wherein the operational action is presented verbally, integrated with the image data, or presented separately on a display.

13. The system of claim 9, wherein the adjustments to operations of one or more medical devices are automatically implemented.

14. The system of claim 9, wherein the current contextual data includes one or more of: a phase of the medical procedure, tissue response of a patient undergoing the medical procedure, an anatomical structure of the patient, environmental conditions of the medical procedure, and conditions of medical instruments involved in the medical procedure.

15. The system of claim 9, wherein the adjustments to operations of one or more medical devices include one or more of: adjusting operational settings used during the medical procedure, overlaying information on image data presented by the one or more medical devices, and performing different operational functions.
